(19) United States
(12) Patent Application Publication
(10) Pub. No.: US 2010/0015259 A1
(43) Pub. Date: Jan. 21, 2010

(54) Composition and method for improving human concentration, memory and other cognitive brain

(76) Inventor: Jerry J. Palmisano, Pittsburgh, PA (US)

Correspondence Address: Gary P. Topolosky, 4031 Brownsville Road, Pittsburgh, PA, 15227-3419, US

(21) Appl. No.: 12/460,203

(22) Filed: Jul. 15, 2009

Related U.S. Application Data
(60) Provisional application No. 61/135,088, filed on Jul. 16, 2008.

(51) Int. Cl. A61K 36/16; A61P 25/00

(52) U.S. Cl. 424/752

(57) ABSTRACT

This formulaic composition has synergistic effects on the mental function of humans including reduction of mental fatigue, maintenance and enhancement of concentration and mental vigor. The main components are theanine, at levels in excess of 200 mg/dose, and Gingko Biloba extract. Further additions include small amounts of Vitamins B₁, B₂, B₅, B₆ and C as well as some L-Lysine Monohydrochloride. The composition has shown improvements for individuals diagnosed with attention deficit disorder (ADD) and mild autism. Related methods of use are also disclosed.

### CROSS-REFERENCE TO RELATED APPLICATIONS

This application claims priority to U.S. Provisional Application Ser. No. 61/135,088, filed on Jul. 16, 2008, the disclosure of which is fully incorporated by reference.

### BACKGROUND OF THE INVENTION

1. Field of the Invention

This invention relates to a composition for improving the cognitive brain function of humans. Particularly, this composition improves human concentration, memory and resistance to mental fatigue. The invention provides nutritional assistance for those struggling with poor mental focus like those suspected of having, or diagnosed with, Attention Deficit Disorder (or “ADD”). Still other benefits are observed with mild cases of autism and related maladies. A related method is also disclosed.

2. Relevant Art

There are numerous dietary supplements with varying lists of components. Some propose solutions for problems similar to those addressed by the present invention. Representative of these prior disclosures are:

Ozeki U.S. Pat. No. 7,273,621 disclosed using a capsule with sugar and L-theanine powder for “promoting” sleep in humans. Meyrowitz U.S. Pat. No. 7,238,373 claimed a nutritional supplement for detoxification that includes among its many elemental components Vitamins C, B₆ and B₅.

Walsh U.S. Pat. No. 7,232,575 disclosed a method for “treating” autism with a nutrient supplement that included Vitamin C, Vitamin B₆ and lysine. Shoemake U.S. Pat. No. 7,112,343 used a combination of all natural herbs for immune system restoration. One of the elements therein was a gingko leaf.

Okubo U.S. Pat. No. 7,094,787 used up to 200 mg theanine for suppressing or moderating smoking urges. In Grose U.S. Pat. No. 7,029,708, there was claimed a combination of B vitamins and Gingko Biloba, among other additives, for improving estrogen deficiencies.

Gorsek U.S. Pat. No. 6,649,195 employed a combination of Vitamin C, Gingko Biloba and many other additives for human eyesight enhancement and maintenance. Yet another natural substance preparation combined Vitamin B₆ and Gingko Biloba extract with other additives to affect a variety of physiological improvements noted at columns 11 and 12 of Stuckler U.S. Pat. No. 6,605,296.

In Ueda U.S. Pat. No. 6,589,566, purified theonine was used to reduce the metal taste of several, duly noted mineral compositions. And Lang U.S. Pat. No. 6,541,043 added Vitamins B₅, B₆ and C to many other components for specifically treating ADD and hyperactivity. Per claims 9 and 14, Gingko is one of the “optional” additives.

In Nozawa U.S. Pat. No. 6,462,051, there is disclosed a composition for reducing mental fatigue. It supposedly maintains and enhances concentration with 50 mg of L-theanine, caffeine and arginine. Additionally, Vitamins C, B₁, B₂, and B₆ may be added.

Gingko Biloba and L-theanine were combined with several herbs to make the sexual response enhancing composition of Heleen U.S. Pat. No. 6,444,237. And for Foreman U.S. Pat. No. 6,399,114, Vitamins C, B₁, B₂, B₆ and Gingko Biloba were combined with other components to treat “nervous system disorders” including autism and ADD.

Duckett U.S. Pat. No. 6,340,480 devised a composition for promoting circulation and healing human skin. In alternate embodiments, Gingko Biloba (claim 7) or L-Lysine (claim 10) could be added to the main elements of arginine, ginseng and Ziyphi fructus.

In Kakuda U.S. Pat. No. 6,271,259, an “effective amount” of theanine was dissolved in a salt or glucose solution for treating individuals suffering from Parkinson's disease. Castillo U.S. Pat. No. 6,264,994 combined Gingko Biloba with other plant-based derivatives for treating Alzheimer's and other amyloidoses.

Altman U.S. Pat. No. 6,159,986 combined Vitamin B with huperzine and hypericin, to inhibit human memory loss. More broadly, Bell U.S. Pat. No. 5,902,797 suggested a nutritional approach with carbohydrates, proteins and fats to address the low appetite, weight losses that result from some ADD treatments.

Kakuda U.S. Pat. No. 5,736,575 incorporated small amounts of theanine into an “excitement accelerating agent”. And the product sold as “Focus Factor” is covered by Smith U.S. Published Application No. 2005-244,510. Finally, in Heuer U.S. Published Application No. 2007-237,786, about 52 mg of theanine may be added to a supplement for: “rapid weight loss, controlling appetite, managing stress, supporting relaxation, combating fatigue and supporting mental well-being”.

### SUMMARY OF THE INVENTION

Known symptoms of mental fatigue include lack of concentration, emotional instability, sleep disturbance, and the like. While physical fatigue can be treated with sufficient rest, a full recovery from mental fatigue is usually not possible with rest alone. What is needed is a synergistic combination of materials that will positively impact one's mental function without otherwise harming the long or short term health of that individual.

The positive influence of nutritional, dietary ingredients on energy production, neurophysiology, and neurotransmission cannot be understated. Various combinations of constituents should be investigated to find what best grouping improves the learning and memory skills to recipients of all ages.

Preferred formulae of the present invention offer a unique combination of cognitive support. They reduce mental fatigue while also maintaining or enhancing concentration and mental vigor. These dietary formulae improve individual attentiveness through the efficient formation of mental energy and synthesis of key neurotransmitters typically associated with memory, focus, concentration and mood.

Main components of this dietary supplement are the addition of theanine levels above about 200 mg, typically above 250-300 mg per dose. Most preferably, the composition of this invention includes about 400 mg or more of L-Theanine. The other main constituent is the naturally generated additive Ginkgo Biloba extract. Preferred levels of this exceed 10 mg per dose. More preferably, about 15-50 mg of Ginkgo Biloba are combined with the aforementioned theanine additive. Most preferably, each dose of this invention includes about 20 to 25 mg of Ginkgo Biloba 24/6 extract.

Several secondary elements are preferably combined with the main components of theanine and Ginkgo Biloba (above). These are known vitamin additives but at atypically high concentration levels (considering the Daily Values expected for same with a 2000 calorie diet). For instance, preferred compositions of this invention would include more than 25 mg of Vitamin C, preferably about 30-70 mg of same, and most preferably around about 45-60 mg per dose.

In addition, preferred formulae of this invention include greater than 5 mg each of Vitamins B₁, B₂ and B₆. More preferably, these B-vitamins are present at levels of about 7-25 mg/dose for Vitamin B₁; about 10-30 mg/dose for Vitamin B₂ and about 15-40 mg/dose for Vitamin B₆. Additional benefits are observed with yet another Vitamin B component. Particularly, about 2-10 mg of Vitamin B₅ should be present. More particularly, about 4-6 mg/dose of Vitamin B₅ should be included.

Yet another secondary element would be L-Lysine Monohydrochloride, FCC. Preferred levels of this should be between about 10-50 mg/dose; with most preferred levels being between about 15-30 mg/dose.

To best deliver the foregoing combination of elements in a single dose, they may be combined with known medicinal delivery additives including but not limited to: rice flour, magnesium stearate, silica and microcrystalline cellulose, or “MCC”.

### DETAILED DESCRIPTION OF PREFERRED EMBODIMENTS

### I. General Description

For every numerical range set forth, it should be noted that all numbers within the range, including every fraction or decimal between its stated minimum and maximum, are considered to be designated and disclosed by this description. As such, herein disclosing a preferred range of about 250-600 mg/dose of theanine expressly includes theanine levels of about 255, 275, 290 and 300 mg/dose . . . and so on, up to about 525, 555, 575 and 590 mg/dose.

Unless defined otherwise, all technical and scientific terms used herein have the same meaning as commonly understood by one of ordinary skill in the art to which this invention belongs. Although any methods and materials similar or equivalent to those described herein can be used in the practice or testing of this invention, the preferred methods and materials are described herein.

The current inventions in this application are in the fields of nutritional supplements and methods of using such supplements to improve and maintain memory, focus, concentration and mood.

The following detailed description and examples provide representative results that can be used by a skilled artisan to prepare and administer such compositions. This description is given for clearness and understanding only and no unnecessary limitations should be understood from same.

The preferred formulaic composition of this invention consists primarily of the following main ingredients: greater than 200 mg/dose of L-theanine and Gingko Biloba extract. Secondary additives thereto include Vitamins B₁, B₂, B₅ and B₆ as well as some Vitamin C and L-Lysine Monohydrochloride, FCC. Each of these components is more clearly defined below.

### II. Components of the Composition

Theanine—The theanine used in this invention can be at least one member selected from the group consisting of: L- or D-glutamic acid γ-alkylamide, such as L-glutamic acid γ-ethylamide (L-theanine), L-glutamic acid γ-methylamide, D-glutamic acid γ-ethylamide (D-theanine), D-glutamic acid γ-methylamide, and/or derivatives having an L- or D-glutamic acid γ-alkylamide group as the basic structure (for example, glycosides of L- or D-glutamic acid γ-alkylamide and others). Among them, L-theanine is especially preferable from the viewpoint of availability and safety. It can be obtained from natural sources and it is also approved as a food additive.

Theanine can be obtained by various methods already known. Concretely, theanine can be obtained by biosynthesis using methods in which plants or microorganisms are cultured, by extraction from tea leaves, and also by fermentation or chemical synthesis.

To achieve the mental improvements of this invention, a dosage of dietary supplement should have theanine levels above about 200 mg, typically above 250-300 mg per dose, up to about 600 or 650 mg/dose. Most preferably, the composition of this invention includes about 400 mg or more of L-Theanine.

Gingko Biloba—Extracts of the other main supplement additive are preferably derived from the leaves of a Gingko Biloba tree. Many commercially available brands of same are available. Gingko Biloba extracts are present in the composition of this invention at levels in excess of 10 mg per dose. Preferably, about 15-50 mg of Ginkgo Biloba are combined with the aforementioned theanine additive. Most preferably, each dose of this invention includes about 20 to 25 mg of Ginkgo Biloba 24/6 extract.

B-Complex Vitamins—Among the most important nutrients known to maintain optimal mental function are B-complex vitamins. These nutrients play both direct and indirect roles in neurological function. Indirectly, B vitamins assist with methylation, the process by which toxic byproducts of cellular metabolism are removed from the body. Methylation is the body's chief mechanism for detoxification. When this process goes awry, there can be a buildup of toxic homocysteine. Elevated homocysteine is shown to result in decreased neural and systemic oxygenation, increased free radical pathology, accelerated arteriosclerosis, cancer, neuro-vascular decline, and neuro-degenerative disorders. It is also a marker for memory loss, cognitive dysfunction and Alzheimer disease. The process of methylation can be optimized, and toxic levels of homocysteine reduced, by consuming specific B-complex vitamins. In addition, studies show that people with a higher blood nutriture of B-complex vitamins score higher on cognitive function tests.

Vitamin B₁ (thiamin) was the first of the B vitamins to be discovered. It is required for the production of multiple enzymes that are necessary for the conversion of glucose into energy in the brain. This nutrient also mimics the activities of acetylcholine, the major learning neurotransmitter associated with attention, concentration and memory. Increased thiamin consumption is associated with improved cognitive function and faster reaction times, with participants subjectively describing supplementation effects as “feeling clearheaded, composed, and energetic”. Vitamin B₁ may be present in the overall composition as thiamin HCL, preferably from about 7 to 25 mg/dose, more preferably from about 10 to 20 mg, and even more preferably around about 15 mg per dose. At the latter level, Vitamin B₁ would be administered at about 1000 times the daily value level for a 2000 calorie diet.

Vitamin B₂ (riboflavin) is a water-soluble, B vitamin and primarily found in the body as an integral component of the coenzymes, flavin adenine dinucleotide (FAD) and flavin mononucleotide (FMN). Coenzymes derived from riboflavin are termed flavocoenzymes, and enzymes that use a flavocoenzyme are called flavoproteins Living organisms derive most of their energy from oxidation-reduction (redox) reactions or processes that involve the transfer of electrons. Flavocoenzymes participate in redox reactions in numerous metabolic pathways. They are critical for the metabolism of carbohydrates, fats, and proteins. FAD, in particular, is part of the electron transport (respiratory) chain central to energy production. In conjunction with cytochrome P-450, flavocoenzymes also participate in the metabolism of drugs and toxins.

Vitamin B₂ may be present in the overall composition as riboflavin, preferably from about 10 to 30 mg/dose, more preferably from about 15 to 25 mg, and even more preferably around about 20 mg per dose. At the latter level, Vitamin B₂ would be administered at nearly 1200 times the daily value level for a 2000 calorie diet.

Vitamin B₅ (p-antothenic acid) is an ideal secondary additive as it must be present for the synthesis of acetylcholine. The present composition may contain Vitamin B₅ as calcium pantothenate and/or pantethine, preferably as d-calcium pantothenate. Vitamin B₅ may comprise preferably from about 2 to 10 mg/dose, more preferably from about 4 to 6 mg and most preferably around about 5 mg/dose. At the latter level, Vitamin B₅ would be administered at around 50% of the daily value level for same with a 2000 calorie diet.

Vitamin B₆ (pyridoxine) is required in the production of the neurotransmitters like norepinephrine, serotonin and dopamine. It has been shown to enhance memory and cognitive function. Low levels of this nutrient correspond to poor scores on cognitive function tests and premature neural aging. The Vitamin B₆ in the present compositions may be either a pyridoxine, pyridoxal or pyridoxamine. Preferably, Vitamin B₆ is present in the composition as pyridoxine HCL. In general, Vitamin B₆ comprises preferably about 15 to 40 mg/dose, more preferably about 20 to 30 mg, and even more preferably around about 25 mg per dose. At the latter level, Vitamin B₆ would be administered at 1250% of its daily value level for a 2000 calorie diet.

Additional Secondary Components—Vitamin C plays multiple roles in cognitive function. It is so important to neural tissue that concentrations of Vitamin C are fifteen times higher in the brain than elsewhere in the body. It is also involved in the production of several neurotransmitters, including acetylcholine, dopamine and norepinephrine. Administration of this nutrient has been shown to increase IQ points, and its use to treat memory disorders is currently being explored. Vitamin C is present in the preferred compositions of this invention at more than 25 mg/dose. Preferably, Vitamin C is present as ascorbic acid. Alternative forms for same include sodium ascorbate, ascorbyl palmitate, calcium ascorbate, potassium ascorbate and/or zinc ascorbate. Vitamin C preferably comprises about 30 to 70 mg, more preferably about 45 to 60 mg, and even more preferably around about 50 mg per dose. At the latter level, Vitamin C would be administered at about 83% of its daily value level for a 2000 calorie diet.

L-Lysine Monohydrochloride FCC is a recommended amino acid addition to the formulae of this invention. L-lysine provides nutritional support for the body's natural defenses and is particularly helpful in restoring normal arginine levels. Preferred levels of this additive should be between about 10 to 50 mg/dose; with most preferred levels being between about 15-30 mg/dose.

The exact proportions of the above-disclosed components may vary depending upon the concentration of active ingredients in the compositions and the desire to optimize bioavailability of these constituents therein. Using the guidance provided above and a basic knowledge of drug preparation and pharmacology, one skilled in the art could easily adjust the proportions of the separate components of the composition so as to obtain a composition which has the therapeutic affects discussed above and shown herein. The discussion of the proportions of ingredients in the composition provided above is merely meant as an example and is not intended to limit the scope of the present invention from including any novel combination of the disclosed components for the purposes prescribed hereinabove.

Others—In addition to the above primary and secondary components, the present composition may contain still other nutrients or herbal extracts that assist, directly or indirectly, in maintaining or restoring mental health by improving the memory, focus, concentration or mood of a patient.

One example of preferred formulation for this invention is provided in following Table 1 below.

TABLE 1

| Component | Amount per serving | % Daily Value |
|---|---|---|
| Vitamin C | 50 mg | 83 |
| Vitamin B₆ | 25 mg | 1250 |
| Vitamin B₂ | 20 mg | 1176 |
| Vitamin B₁ | 15 mg | 1000 |
| Vitamin B₅ | 5 mg | 50 |
| L-Theanine | 400 mg | n/a |
| Gingko Biloba | 25 mg | n/a |
| L-Lysine | 20 mg | n/a |
| Monohydrochloride | | |

### III. Preparation of the Compositions

The compositions of this invention are preferably provided as capsules, with a single dose of one capsule to be taken 1 to 3 times each day. As dry powders, these formulae can be made by freeze drying, spray drying or the like. Still other versions include solutions, tablets, granules, suspensions, emulsions, ampules and/or injections.

The ingredients may be compounded, for example, with the usual non-toxic pharmaceutically acceptable carriers for capsules, tablets, pellets, and the like. Formulations of the present invention encompass various combinations of rice flour, ascorbyl palmitate, silica and microcrystalline cellulose (or MCC). Still other carrier components include, but are not limited to: water, talc, glucose, lactose, gum acacia, gelatin, mannitol, starch paste, magnesium trisilicate, corn starch, keratin, potato starch, and urea. In addition, auxiliary, stabilizing, thickening, flavoring/coloring agents and perfumes may be added as desired.

For preparing solid compositions such as capsules or tablets, the principal ingredients are mixed with a carrier ( e.g., conventional ingredients such as corn starch, lactose, sucrose, sorbitol, talc, stearic acid, magnesium stearate, dicalcium phosphate or gums) and other diluents (e.g., water) to form a solid pre-formulation composition containing a substantially homogeneous mixture of the aforesaid compositions, or a non-toxic salt thereof. When referring to pre-formulation compositions as substantially homogenous, it is meant that the active ingredients are dispersed evenly throughout the compositions so that the compositions may be readily subdivided into equally effective unit dosage forms such as capsules, tablets and pills. These solid pre-formulation compositions are then subdivided into unit dosage forms of the type described above containing predetermined amounts of the compositions of the present invention, preferably in capsules, tablets or pills.

The capsules, tablets or pills of this invention can be coated or otherwise compounded to provide a dosage form that affords the advantage of prolonged action. For example, the capsules, tablets or pills can comprise both an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

While the invention has been described in connection with what is presently considered to be the most practical and preferred embodiment, it is to be understood that the invention is intended to cover various modifications and equivalent arrangements included within the spirit and scope of the appended claims.

What is claimed is:

1. A composition for reducing mental fatigue, maintaining and enhancing concentration and mental vigor comprises as its main components a mixture with more than about 200 mg of theanine and at least about 15 mg of Gingko Biloba extract per dosage.

2. The composition of claim 1 which further comprises at least one B-complex Vitamin selected from the group consisting of Vitamin B₁, Vitamin B₂, Vitamin B₅ and Vitamin B₆.

3. The composition of claim 2 which comprises at least about 5 mg each of Vitamin B₁, Vitamin B₂, Vitamin B₅ and Vitamin B₆.

4. The composition of claim 3 which comprises at least about 10 mg of Vitamin B₁.

5. The composition of claim 3 which comprises at least about 15 mg of Vitamin B₂.

6. The composition of claim 3 which comprises at least about 20 mg of Vitamin B₆.

7. The composition of claim 1 which further comprises at least about 30 mg of Vitamin C.

8. The composition of claim 1 which further comprises at least about 10 mg of L-Lysine Monohydrochloride.

9. The composition of claim 1 which contains more than about 300 mg of L-theanine.

10. The composition of claim 9 which contains at least about 400 mg of L-theanine.

11. The composition of claim 1, wherein each dose contains the formulaic composition of TABLE 1.

12. A dietary supplement for treating attention deficit disorder and mild cases of autism, said supplement comprising a composition with more than about 300 mg of theanine and at least about 15 mg of Gingko Biloba extract per dose.

13. The dietary supplement of claim 12 which further comprises at least one B-complex Vitamin selected from the group consisting of Vitamin B₁, Vitamin B₂, Vitamin B₅ and Vitamin B₆.

14. The dietary supplement of claim 13 which comprises at least about 5 mg each of Vitamin B₁, Vitamin B₂, Vitamin B₅ and Vitamin B₆.

15. The dietary supplement of claim 12 which further comprises at least about 30 mg of Vitamin C and at least about 10 mg of L-Lysine Monohydrochloride.

16. The dietary supplement of claim 12 which contains at least about 400 mg of L-theanine.

17. The dietary supplement of claim 12, wherein each dose contains about 50 mg of Vitamin C, about 25 mg of Vitamin B₆, about 20 mg of Vitamin B₂, about 15 mg. of Vitamin B₁, about 5 mg of Vitamin B₅, about 400 mg of L-Theanine, about 25 mg of Gingko Biloba and about 20 mg of L-Lysine Monohydrochloride.

18. A method for improving mental performance comprises administering a daily supplement consisting of one to three capsules of a composition containing about 50 mg of Vitamin C, about 25 mg of Vitamin B₆, about 20 mg of Vitamin B₂, about 15 mg. of Vitamin B₁, about 5 mg of Vitamin B₅, about 400 mg of L-Theanine, about 25 mg of Gingko Biloba and about 20 mg of L-Lysine Monohydrochloride.

19. A method for treating attention deficit disorder comprises administering a daily supplement consisting of one to three capsules of a composition containing about 50 mg of Vitamin C, about 25 mg of Vitamin B₆, about 20 mg of Vitamin B₂, about 15 mg. of Vitamin B₁, about 5 mg of Vitamin B₅, about 400 mg of L-Theanine, about 25 mg of Gingko Biloba and about 20 mg of L-Lysine Monohydrochloride.

20. A method for treating mild autism comprises administering a daily supplement consisting of one to three capsules of a composition containing about 50 mg of Vitamin C, about 25 mg of Vitamin B₆, about 20 mg of Vitamin B₂, about 15 mg. of Vitamin B₁, about 5 mg of Vitamin B₅, about 400 mg of L-Theanine, about 25 mg of Gingko Biloba and about 20 mg of L-Lysine Monohydrochloride.
